(19) United States
(12) Patent Application Publication
(10) Pub. No.: US 2012/0167286 A1
(43) Pub. Date: Jul. 5, 2012

(54) Structure of Swimming Goggles

(76) Inventor: Xia-Sheng CHEN, New Taipei City (TW)

(21) Appl. No.: 13/339,754

(22) Filed: Dec. 29, 2011

(30) Foreign Application Priority Data
Dec. 29, 2010 (TW) 099225377

(51) Int. Cl. A61F 9/02

(52) U.S. Cl. 2/439

(57) ABSTRACT

The present invention relates to a goggle, which comprise two lenses, with a hole or concave at the periphery or near the external; a frame body, with front-end protrusions used to withhold the holes or the concave of said lenses; and a headband, with buckles on both sides, used to connect with the two sides of said frame body. With above said design and structure, when users' myopia changing or the lenses being damaged, users can combine or disassembly the lenses and the frame body of the goggle easily, in order to achieve the purpose for economic and environmental protection.

### BACKGROUND OF THE INVENTION

1. Field of the Invention

The present invention relates to a goggle structure, particularly it pertains to a goggle cable of letting users can combine or disassembly the lenses and the frame body of the goggle easily when users' myopia changing or the lenses being damaged, in order to achieve the purpose for economic and environmental protection.

2. Description of the Related Art

Swimming is cable of improving lung capacity, and also exercising the muscles. It a great help for physical health. While swimming, the body will be affected by the buoyancy of water that the body burden is greatly reduced. It is quite suitable for patient rehabilitation. But the eyes in the water there will be pain, blurred vision and other issues, it must be through the goggle as aids to enjoy the fun of diving or swimming.

However, many people have myopia, astigmatism and other eye diseases, whenever vision changes or when lenses wear or damage to, have to re-purchase another pair of goggle, not only to spend money but also a waste of resources.

### SUMMARY OF THE INVENTION

In view of the imperfections of conventional goggles, the inventor of the present invention has spent years researching and developing a goggle cable of letting users can combine or disassembly the lenses and the frame body of the goggle easily when users' myopia changing or the lenses being damaged, in order to achieve the purpose for economic and environmental protection.

The present invention comprise two lenses, with a hole or concave at the periphery or near the external; a frame body, with front-end protrusions used to withhold the holes or the concave of said lenses; and a headband, with buckles on both sides, used to connect with the two sides of said frame body.

The main aim of the present invention is to provide a goggle cable of letting users can combine or disassembly the lenses and the frame body of the goggle easily when users' myopia changing or the lenses being damaged, in order to achieve the purpose for economic and environmental protection.

These and other objects, features and advantages of the present invention will become more apparent from the following description and the appended claims, taken in connection with the accompanying drawings in which preferred embodiment of the present invention are shown by way of illustrative example.

### BRIEF DESCRIPTION OF THE DRAWINGS

FIG. 1 is a diagram showing the first embodiment of the present invention.

FIG. 2 is a diagram showing the second embodiment of the present invention.

FIG. 3 is a diagram showing the third embodiment of the present invention.

### DETAILED DESCRIPTION OF THE PREFERRED EMBODIMENT

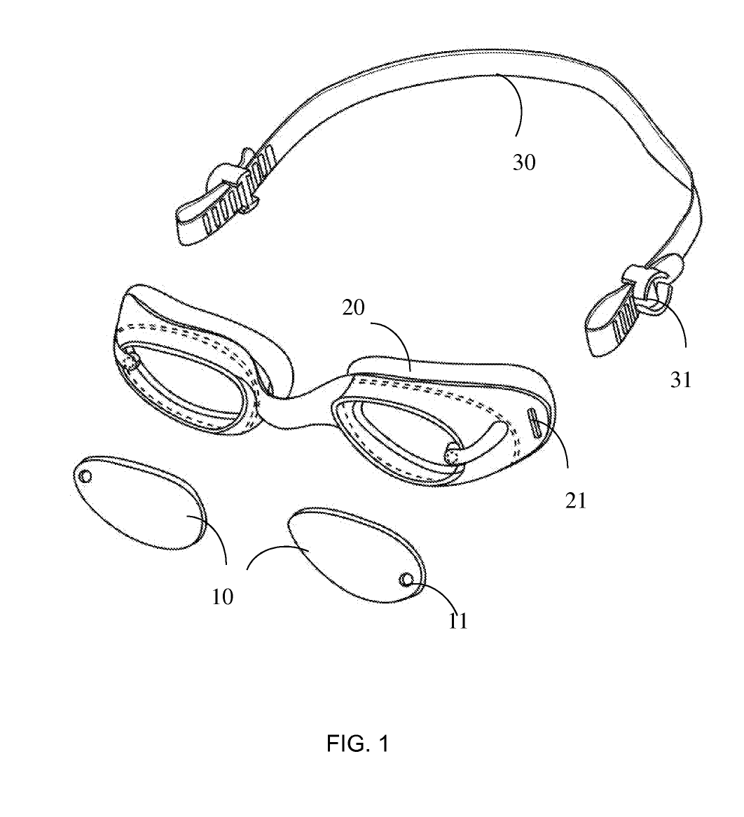

Please refer to FIG. 1, FIG. 1 is a diagram showing the first embodiment of the present invention. The present invention comprise: two lenses 10, with a hole 11 or concave at the periphery or near the external; a frame body 20, with front-end protrusions 21 used to withhold the holes 11 or the concave of said lenses 10; and a headband 30, with buckles 31 on both sides, used to connect with the two sides of said frame body 20.

With above said design and structure, when users' myopia changing or the lenses 10 being damaged, users can combine or disassembly the lenses 10 and the frame body 20 of the goggle easily, in order to achieve the purpose for economic and environmental protection.

It is worth mentioning that said frame body 20 could comprise a support part for the nose that it makes said frame body 20 being bendable; the protrusions 21 of said frame body 20 could be latches, screws or fixed bolt; said headband 30 could be flexible and configured with an adjusting element; said headband 30 and said frame body 20 could be integrated; said headband 30 and said frame body 20 could be connected with latches, buckles, screws or fixed bolt; a gasket could be configured between said frame body 20 and said lenses 10 to increase watertight; and said frame body 20 and said support part for the nose could be integrated.

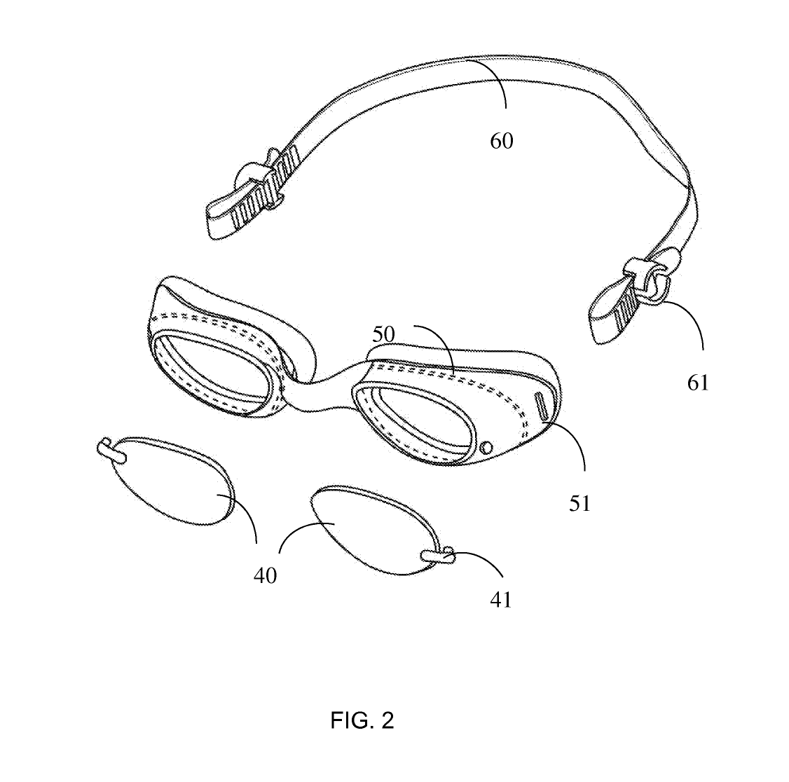

Please refer to FIG. 2, FIG. 2 is a diagram showing the second embodiment of the present invention. The present invention comprise: two lenses 40, with protrusions 41 at the periphery or near the external; a frame body 50, with front-end holes 51 or concave used to be withhold by the protrusions 41 of said lenses 40; and a headband 60, with buckles 61 on both sides, used to connect with the two sides of said frame body 50.

With above said design and structure, when users' myopia changing or the lenses 40 being damaged, users can combine or disassembly the lenses 40 and the frame body 50 of the goggle easily, in order to achieve the purpose for economic and environmental protection.

It is worth mentioning that said frame body 50 could comprise a support part for the nose that it makes said frame body 50 being bendable; the protrusions 41 of said lenses 40 could be latches, screws or fixed bolt; said headband 60 could be flexible and configured with an adjusting element; said headband 60 and said frame body 50 could be integrated; said headband 60 and said frame body 50 could be connected with latches, buckles, screws or fixed bolt; a gasket could be configured between said frame body 50 and said lenses 40 to increase watertight; and said frame body 50 and said support part for the nose could be integrated.

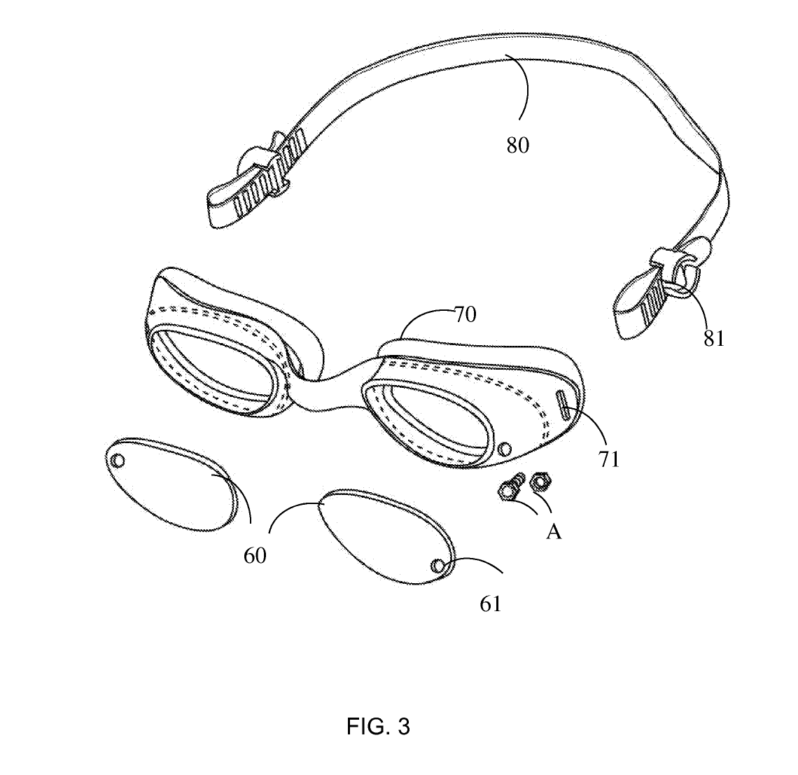

Please refer to FIG. 3, FIG. 3 is a diagram showing the third embodiment of the present invention. The present invention comprise: two lenses 70, with a fastener 71 at the periphery or near the external; a frame body 80, with front-end fasteners 81 used to withhold the fasteners 71 of said lenses 70; and a headband 90, with buckles 91 on both sides, used to connect with the two sides of said frame body 80.

With above said design and structure, when users' myopia changing or the lenses 70 being damaged, users can combine or disassembly the lenses 70 and the frame body 80 of the goggle easily, in order to achieve the purpose for economic and environmental protection.

It is worth mentioning that said frame body 80 could comprise a support part for the nose that it makes said frame body 80 being bendable; said headband 90 could be flexible and configured with an adjusting element; said headband 90 and said frame body 80 could be integrated; said headband 90 and said frame body 80 could be connected with latches, buckles, screws or fixed bolt; a gasket could be configured between said frame body 80 and said lenses 70 to increase watertight; and said frame body 80 and said support part for the nose could be integrated.

As is understood by a person skilled in the art, the foregoing preferred embodiment of the present invention is an illustration, rather than a limiting description of the present invention. It is intended to cover various modifications and similar arrangements, for example, the material of goggle, the position of the hole, the position of the concave, the position of the protrusions, and the structure for fasteners etc., all the above may vary and should be considered within the spirit and scope of the appended claims of the present invention. In short, the spirit and scope should be accorded the broadest interpretation so as to encompass all such modifications and similar structures.

1. A goggle comprising:
two lenses, with a hole or concave at the periphery or near the external;
a frame body, with front-end protrusions used to withhold the holes or the concave of said lenses; and
a headband, with buckles on both sides, used to connect with the two sides of said frame body;
with above said design and structure, when users' myopia changing or the lenses being damaged, users can combine or disassembly the lenses and the frame body of the goggle easily, in order to achieve the purpose for economic and environmental protection.

2. The goggle as in claim 1, wherein said frame body could comprise a support part for the nose that it makes said frame body being bendable.

3. The goggle as in claim 1, wherein the protrusions of said frame body could be latches, screws or fixed bolt.

4. The goggle as in claim 1, wherein said headband could be flexible and configured with an adjusting element.

5. The goggle as in claim 1, wherein said headband and said frame body could be integrated.

6. The goggle as in claim 1, wherein said headband and said frame body could be connected with latches, buckles, screws or fixed bolt.

7. The goggle as in claim 1, wherein a gasket could be configured between said frame body and said lenses to increase watertight.

8. The goggle as in claim 2, wherein said frame body and said support part for the nose could be integrated.

9. A goggle comprising:
two lenses, with protrusions at the periphery or near the external;
a frame body, with front-end holes or concave used to be withhold by the protrusions of said lenses; and
a headband, with buckles on both sides, used to connect with the two sides of said frame body;
with above said design and structure, when users' myopia changing or the lenses being damaged, users can combine or disassembly the lenses and the frame body of the goggle easily, in order to achieve the purpose for economic and environmental protection.

10. The goggle as in claim 9, wherein said frame body could comprise a support part for the nose that it makes said frame body being bendable.

11. The goggle as in claim 9, wherein the protrusions of said lenses could be latches, screws or fixed bolt.

12. The goggle as in claim 9, wherein said headband could be flexible and configured with an adjusting element.

13. The goggle as in claim 9, wherein said headband and said frame body could be integrated.

14. The goggle as in claim 9, wherein said headband and said frame body could be connected with latches, buckles, screws or fixed bolt.

15. The goggle as in claim 9, wherein a gasket could be configured between said frame body and said lenses to increase watertight.

16. The goggle as in claim 10, wherein said frame body and said support part for the nose could be integrated.

17. A goggle comprising:
two lenses, with a fastener at the periphery or near the external;
a frame body, with front-end fasteners used to withhold the fasteners of said lenses; and
a headband, with buckles on both sides, used to connect with the two sides of said frame body;
with above said design and structure, when users' myopia changing or the lenses being damaged, users can combine or disassembly the lenses and the frame body of the goggle easily, in order to achieve the purpose for economic and environmental protection.

18. The goggle as in claim 17, wherein said frame body could comprise a support part for the nose that it makes said frame body being bendable.

19. The goggle as in claim 17, wherein said headband could be flexible and configured with an adjusting element.

20. The goggle as in claim 17, wherein said headband and said frame body could be integrated.

21. The goggle as in claim 17, wherein said headband and said frame body could be connected with latches, buckles, screws or fixed bolt.

22. The goggle as in claim 17, wherein a gasket could be configured between said frame body and said lenses to increase watertight.

23. The goggle as in claim 18, wherein said frame body and said support part for the nose could be integrated.
